(19) United States
(12) Patent Application Publication
(10) Pub. No.: US 2021/0145861 A1
(43) Pub. Date: May 20, 2021

(54) CHITOSAN CONTAINING COMPOSITIONS AND METHODS RELATING TO SAME

(71) Applicant: Gregory J. PAMEL, New York, NY (US)

(72) Inventor: Gregory J. PAMEL, New York, NY (US)

(21) Appl. No.: 16/950,359

(22) Filed: Nov. 17, 2020

Related U.S. Application Data
(60) Provisional application No. 62/936,791, filed on Nov. 18, 2019.

(51) Int. Cl. A61K 31/722; A61K 31/525; A61K 31/14; A61K 45/06; A61K 9/00; A61K 31/675; A61K 31/7084

(52) U.S. Cl. CPC A61K 31/722; A61K 31/525; A61K 31/14; A61K 31/7084; A61K 9/0048; A61K 31/675; A61K 45/06

(57) ABSTRACT

Compositions containing chitosan and a cationic polysaccharide or functionalized cationic polysaccharide and methods for using these compositions to treat corneal transplant are described herein. The compositions can be applied to the eye of a patient in need of treatment to penetrate the corneal epithelial tissue and improve crosslinking without having removed the epithelium.

### CROSS-REFERENCE TO RELATED APPLICATIONS

This application claims priority from U.S. Provisional No. 62/936,791 entitled “Composition Containing Chlorine Dioxide and Methods for Using Same,” filed Nov. 18, 2019.

### GOVERNMENT INTERESTS

Not applicable

### PARTIES TO A JOINT RESEARCH AGREEMENT

Not applicable

### INCORPORATION OF MATERIAL ON COMPACT DISC

Not applicable

### BACKGROUND

Corneal scarring is a major cause of blindness, especially in developing countries. There are various causes for corneal scarring, which include: bacterial infections, viral infections, fungal infections, parasitic infections, genetic corneal problems, Fuch's dystrophy, and other corneal dystrophies. A corneal transplant is often required if the corneal scarring is extensive and cannot be corrected by other means. However, there can be major complications associated with a corneal transplant, such as corneal graft rejection wherein the transplanted cornea is rejected by the patient's immune system. Methods for corneal transplantation that reduce the likelihood that the implanted cornea will be rejected by the patient are needed.

### SUMMARY OF THE INVENTION

Various embodiments of the invention include compositions containing an effective amount of chitosan, an effective amount of a flavin, and a pharmaceutically acceptable excipient, carrier, or diluent. In some embodiments, the chitosan may be acetylated or deacetylated chitosan, acetylated or deacetylated chitosan salts, acetylated or deacetylated chitin, acetylated or deacetylated chitin sats, acetylated or deacetylated chitooligosaccharides, acetylated or deacetylated chitooligosaccharides salts, acetylated or deacetylated functionalized chitosan derivatives, acetylated or deacetylated functionalized chitosan derivative salts, N,N,N-trimethyl chitosan (TMC), N,N,N-trimethyl-O-(2-hydroxy-3-trimethylammonium propyl)chitosan, N,N,N-trimethyl-O-carboxymethyl chitosan (TMCMC), O-carboxymethyl chitosan (CMC), sodium-form O-carboxymethyl chitosan, hydorgen-form O-carboxymethyl chitosan, N-(2-hydroxy-3-trimethylammonium)propyl chitosan (HTCC), quaternary N-(benzyl)chitosan, and the like combinations thereof. In particular embodiments, an effective amount of chitosan may be about 10% (w/w) to about 75% (w/w) based on the total weight of the composition. In some embodiments, the chitosan may be a thiolated polymer, thiomer, or preactivated thiomer such as, for example, chitosan-cysteine, chitosan-thiogylcolic acid, chitosan-thiobutylamidine, deacetylated gellan gum-cysteine, poly(methacrylic acid)-cysteine, alginate-cysteine, carboxymethycellulose-cysteine, poly(acrylic acid)-cysteine, poly(acrylic acid)-cysteamine, poly(acrylic acid)-homocysteine, and thiolated polycarbophil, and combinations thereof

In some embodiments, the flavin may be riboflavin, riboflavin-5-phosphate, flavin mononucleotide, flavin adenine dinucleotide, flavin guanine nucleotide, flavin cytosine nucleotide, flavin thymine nucleotide, riboflavin free base, phosphorylated riboflavin, riboflavin-5′-phosphate, or salts or derivatives thereof, and the like and combinations thereof. In certain embodiments, an effective amount of flavin may be about 0.005% (w/w) to about 10% (w/w) based on the total weight of the composition.

In some embodiments, the composition may include an effective amount of one or more quaternary ammonium cations such as, for example, benzalkonium chloride, C10-C14-alkyl(ethylbenzyl)dimethylammonium chloride, andor salts or derivatives thereof, and combinations thereof. In some embodiments, the effective amount of one or more quaternary ammonium cations may be 0.00005% (w/w) to about 0.15% (w/w) based on the total weight of the composition.

In some embodiments, the composition may include one or more solvents, co-solvents, buffers, stabilizers, ophthalmic astringents, ophthalmic demulcents, ophthalmic emollients, ophthalmic vasoconstrictors, oxygen-releasing components, activators, hypertonicity agents, and the like and combinations thereof. In particular embodiments, the composition may include one or more anti-inflammatory agents, steroids, anesthetics, antimicrobial agents, chlorine dioxide, and combinations thereof.

Other embodiments are directed to methods for treating corneal disorders by administering an effective amount of a composition comprising chitosan, flavin, and a pharmaceutically acceptable excipient, carrier, or diluent to an eye of a patient in need of treatment. In some embodiments, administration may include periodic or repeated application of the composition to the eye of the patient in need of treatment. In particular embodiments, an effective amount of the composition may be about 10 μl (microliters) to about 200 μl. In some embodiments, the patient in need of treatment has undergone a corneal transplant, has keratoconus, ecstatic corneal disorder, post lasik ectasia, keratectasia, progressive myopia, glaucoma, and combinations thereof.

In some embodiments, the chitosan may be acetylated or deacetylated chitosan, acetylated or deacetylated chitosan salts, acetylated or deacetylated chitin, acetylated or deacetylated chitin sats, acetylated or deacetylated chitooligosaccharides, acetylated or deacetylated chitooligosaccharides salts, acetylated or deacetylated functionalized chitosan derivatives, acetylated or deacetylated functionalized chitosan derivative salts, N,N,N-trimethyl chitosan (TMC), N,N,N-trimethyl-O-(2-hydroxy-3-trimethylammonium propyl)chitosan, N,N,N-trimethyl-O-carboxymethyl chitosan (TMCMC), O-carboxymethyl chitosan (CMC), sodium-form O-carboxymethyl chitosan, hydorgen-form O-carboxymethyl chitosan, N-(2-hydroxy-3-trimethylammonium)propyl chitosan (HTCC), quaternary N-(benzyl)chitosan, and the like combinations thereof. In particular embodiments, an effective amount of chitosan may be about 10% (w/w) to about 75% (w/w) based on the total weight of the composition. In some embodiments, the chitosan may be a thiolated polymer, thiomer, or preactivated thiomer such as, for example, chitosan-cysteine, chitosan-thiogylcolic acid, chitosan-thiobutylamidine, deacetylated gellan gum-cysteine, poly(methacrylic acid)-cysteine, alginate-cysteine, carboxymethycellulose-cysteine, poly(acrylic acid)-cysteine, poly(acrylic acid)-cysteamine, poly(acrylic acid)-homocysteine, and thiolated polycarbophil, and combinations thereof

In some embodiments, the flavin may be riboflavin, riboflavin-5-phosphate, flavin mononucleotide, flavin adenine dinucleotide, flavin guanine nucleotide, flavin cytosine nucleotide, flavin thymine nucleotide, riboflavin free base, phosphorylated riboflavin, riboflavin-5′-phosphate, or salts or derivatives thereof, and the like and combinations thereof. In certain embodiments, an effective amount of flavin may be about 0.005% (w/w) to about 10% (w/w) based on the total weight of the composition.

In some embodiments, the composition may include an effective amount of one or more quaternary ammonium cations such as, for example, benzalkonium chloride, C10-C14-alkyl(ethylbenzyl)dimethylammonium chloride, andor salts or derivatives thereof, and combinations thereof. In some embodiments, the effective amount of one or more quaternary ammonium cations may be 0.00005% (w/w) to about 0.15% (w/w) based on the total weight of the composition.

In some embodiments, the composition may include one or more solvents, co-solvents, buffers, stabilizers, ophthalmic astringents, ophthalmic demulcents, ophthalmic emollients, ophthalmic vasoconstrictors, oxygen-releasing components, activators, hypertonicity agents, and the like and combinations thereof. In particular embodiments, the composition may include one or more anti-inflammatory agents, steroids, anesthetics, antimicrobial agents, chlorine dioxide, and combinations thereof.

### DESCRIPTION OF THE DRAWINGS

Not applicable

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in theirentirety. Nothing herein is to be construed as an admission that the invention is not entitled to antedate such disclosure by virtue of prior invention.

It must also be noted that as used herein and in the appended claims, the singular forms “a,” “an,” and “the” include plural reference unless the context clearly dictates otherwise. Thus, for example, reference to an “amino acid” is a reference to one or more amino acids and equivalents thereof known to those skilled in the art, and so forth.

As used herein, the term “about” means plus or minus 10% of the numerical value of the number with which it is being used. Therefore, about 50% means in the range of 45%-55%.

“Administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering,” when used in conjunction with a chlorine dioxide-containing composition, can include, but is not limited to, providing a chlorine dioxide-containing composition into or onto the target tissue; providing a chlorine dioxide-containing composition systemically to a patient by, e.g., intravenous injection whereby the therapeutic reaches the target tissue. “Administering” a composition may be accomplished by injection, topical administration, or by either method in combination with other known techniques. Such combination techniques include heating, radiation and ultrasound.

The term “animal” as used herein includes, but is not limited to, humans and non-human vertebrates such as wild, domestic, and farm animals.

As used herein, the term “antimicrobial” refers to the ability of the antimicrobial composition described herein to prevent, inhibit or destroy the growth of microbes such bacteria (including Mycobacteria), viruses, fungi, or amoebae

The term “kill” and like terms, refers to the ability of an antimicrobial composition to inhibit or destroy growth of a cellular (e.g., self-replicating) microbe, such as, without limitation, a bacteria or fungus, for example, by reducing a number of colony-forming units of the cellular microbe in a bacteria culture or colony, or to inhibit growth rate of a colony or culture of cells. Likewise, with reference to virus particles or virions (e.g., non-self-replicating), the term “neutralize” refers to a reduction of infectivity of a single virion and to overall infectivity (e.g., a reduction in the number of infectious units (IU) or plaque-forming units (PFU)) of a sample of virus particles). Non-limiting examples of killing of bacteria or fungi, and neutralization of virions, and methods of testing for such killing or neutralization are provided in the Examples below.

The term “inhibiting” includes the administration of a compound of the present invention to prevent the onset of the symptoms, alleviating the symptoms, reducing the symptoms, delaying or decreasing the progression of the infection and/or its symptoms, or eliminating condition or infection.

By “pharmaceutically acceptable,” it is meant the carrier, diluent or excipient must be compatible with the other ingredients of the formulation and not deleterious to the recipient thereof.

As used herein, the term “ophthalmically acceptable” refers to any material or combination of materials which, in the concentrations employed, has no undue detrimental effect on the eye or the ocular tissue with which it comes in contact.

As used herein, the term “therapeutic” means an agent utilized to treat, combat, ameliorate, prevent or improve an unwanted condition or infection of a patient. In part, embodiments of the present invention are directed to the treatment of bacteria (including Mycobacteria), viruses, fungi, or amoebae infections.

A “therapeutically effective amount” or “effective amount” of a composition is a predetermined amount calculated to achieve the desired effect, i.e., to bacteria (including Mycobacteria), viruses, fungi, or amoebae. The activity contemplated by the present methods includes both medical therapeutic and/or prophylactic treatment, as appropriate. The specific dose of a chlorine dioxide containing composition administered according to this invention to obtain therapeutic and/or prophylactic effects will, of course, be determined by the particular circumstances surrounding the case, including, for example, the chlorine dioxide administered, the route of administration, and the condition being treated. The effective amount administered will be determined by the physician in the light of the relevant circumstances including the condition to be treated, the choice of chlorine dioxide to be administered, and the chosen route of administration, and therefore the above dosage ranges are not intended to limit the scope of the invention in any way. A therapeutically effective amount of chlorine dioxide in this invention is typically an amount such that when it is administered in a physiologically tolerable excipient composition, it is sufficient to achieve an effective concentration in the ocular tissue.

The terms “treat,” “treated,” or “treating” as used herein refers to both therapeutic treatment and prophylactic or preventative measures, wherein the object is to prevent or slow down (lessen) an undesired physiological condition or infection, or to obtain beneficial or desired clinical results. For the purposes of this invention, beneficial or desired clinical results include, but are not limited to, alleviation of symptoms; diminishment of the extent of the condition or infection; stabilization (i.e., not worsening) of the state of the condition or infection; delay in onset or slowing of the progression of the condition or infection; amelioration of the condition or infection; and remission (whether partial or total), whether detectable or undetectable, or enhancement or improvement of the condition or infection. Treatment includes eliciting a clinically significant response without excessive levels of side effects.

Generally speaking, the term “tissue” refers to any aggregation of similarly specialized cells which are united in the performance of a particular function.

It is understood, for any of the chemicals of this disclosure, that the chemicals may be in various modified forms such as acetate forms, and sodium phosphate forms, sodium salts, and the like. It is known that any of the reagents mentioned anywhere in this disclosure may be in chemically equivalent forms such as salts, hydrides, esters and other modifications of the basic chemical. For example, dexamethasone in any of the compositions and methods of the invention may be replaced with any of its derivatives, including esters and salts thereof. Examples of such derivatives include, at least, Dexamethasone-17-acetate (CAS RN: 1177-87-3), Dexamethasone Disodium Phosphate (CAS RN: 2392-39-4), Dexamethasone Valerate (CAS RN: 14899-36-6), Dexamethasone-21-isonicotinate (CAS RN: 2265-64-7), Dexamethasone Palmitate (CAS RN: 33755-46-3), Dexamethasone Propionate (CAS RN: 55541-30-5), Dexamethasone Acefurate (CAS RN: 83880-70-0), Dexamethasone-21-galactoside (CAS RN: 92901-23-0), dexamethasone 21-thiopivalate, dexamethasone 21-thiopentanoate, dexamethasone 21-thiol-2-methyl-1-butanoate, dexamethasone 21-thiol-3-methyl-1-butanoate, dexamethasone 21-thiohexanoate, dexamethasone 21-thiol-4-methyl-pentanoate, dexamethasone 21-thiol-3,3-dimethyl-butanoate, dexamethasone 21-thiol-2-ethyl-butanoate, dexamethasone 21-thiooctanoate, dexamethasone 21-thiol-2-ethyl-hexanoate, dexamethasone 21-thiononanoate, dexamethasone 21-thiodecanoate, dexamethasone 21-p-fluorothiobenzoate or a combination thereof.

Various embodiments of the invention are directed to compositions including chitosan and a cationic polysaccharide or functionalized cationic polysaccharide and methods for using such compositions on intact corneal epithelium to enhance penetration of the corneal epithelium. Thus, in some embodiments, the compositions may be combined with an active agent and used to enhance delivery of the active agent to the cornea. In other embodiments, the composition may be used in an epithelium-on corneal collagen crosslinking procedure to improve penetration of crosslinking agents and enhance crosslinking of the collagen to the cornea.

As used herein, the term “chitosan” refers to chitosan and chitosan salts. Chitosan is a deacetylated chitin having the general formula (C₈H₁₃NO₅)ₙ and is a glucosamine polysaccharide. The name chitosan refers to a continuum of soluble polymeric chitin derivatives that can be described and classified according to the fraction of N-acetylated residues (FA) or degree of N-acetylation (DA), the average degree of polymerization (DPn) or the average molecular weight (MWn), the molecular weight distribution (PD, PolyDispersity) and the pattern of N-acetylation (PA) or sequence. The degree of chitosan deacetylation typically ranges from about 55% to about 99%. The chitosan molecular weight typically ranges from about 10,000 Daltons to about 2,000,000 Daltons. Chitosan is non-toxic, biocompatible and biodegradable.

“Chitosan” as used herein encompasses various chitosan derivatives and precursors and combinations of chitosan, chitosan derivatives, and precursors. Chitosan derivatives include, for example, chitin, a linear polysaccharide consisting of β(1→4) linked N-acetyl-D-glucosamine residues and occurs mainly as a structural component in the cell walls of fungi and yeasts and in the exoskeletons of insects and arthropods (e.g., crabs, lobsters and shrimps). Chitosan can be prepared from chitin by partial deacetylation and is a heteropolymer of N-acetyl-D-glucosamine and D-glucosamine residues. Unlike chitin, chitosan is soluble in water or in dilute aqueous acid solutions.

Chitosan derivatives further include chitooligosaccharides (CHOS), which encompass chitopoly- or chitooligo-saccharides that are prepared from chitosan either chemically or enzymatically. Chitosan can be converted to CHOS by acid hydrolysis or by enzymatic hydrolysis with glycosyl hydrolases like chitinases or chitosanases. The FA, w, PD, DPn and PA of the resulting CHOS-mixture depend on the chitosan starting material and the specificity of the enzyme used, as well as on reaction conditions such as reaction time, reaction temperature and reaction pH. Low molecular weight CHOS have been found to be effective antifungal agents.

The amount of chitosan, chitosan derivatives, chitosan precursors, and salts and combinations thereof in the compositions of various embodiments may vary. For example, in some embodiments, the amount of chitosan, chitosan derivatives, chitosan precursors, and salts and combinations thereof may be about 1% (w/w) to about 50% (w/w), about 1% (w/w) to about 30% (w/w), about 1% (w/w) to about 20% (w/w), about 1% (w/w) to about 10% (w/w), about 2% (w/w) to about 30% (w/w), about 2% (w/w) to about 20% (w/w), about 2% (w/w) to about 10% (w/w), about 5% (w/w) to about 50% (w/w), about 5% (w/w) to about 30% (w/w), about 5% (w/w) to about 20% (w/w), about 5% (w/w) to about 10% (w/w), or any range or individual concentration encompassed by these example ranges.

The formulations further include cationic polysaccharides and functionalized cationic polysaccharides. In some embodiments, the formulation may include cationic polysaccharides such as, for example, starch, cellulose, pectin, chitin, chitosan, guar, and the like. In particular embodiments, the cationic polysaccharides may be functionalized with a positively charged moieties that increase the number of amino groups on the cationic polysaccharide or introduce of quaternary ammonium groups, resulting in chitosan derivatives with positive charges over wide pH ranges. Such “functionalized cationic polysaccharides” may be more compatible with host tissues and may be more effective antimicrobial agents. Examples of functionalized cationic polysaccharides include, but are not limited to, N,N,N-trimethyl chitosan (TMC), N,N,N-trimethyl-O-(2-hydroxy-3-trimethylammoniumpropyl)chitosan, N,N,N-trimethyl-O-carboxymethyl chitosan (TMCMC), O-carboxymethyl chitosan (CMC), Na-form CMC, H-form CMC, N-(2-hydroxy-3-trimethylammonium)propyl chitosan (HTCC), quaternary N-(benzyl)chitosan, and the like and combinations thereof.

In certain embodiments, the cationic polysaccharides and functionalized cationic polysaccharides may be flavins such as, for example, riboflavin, riboflavin-5-phosphate, flavin mononucleotide, flavin adenine dinucleotide, flavin guanine nucleotide, flavin cytosine nucleotide, and flavin thymine nucleotide, and in particular embodiments, such formulations may include riboflavin in its free base, phosphorylated, or a salt form. In particular embodiments, the riboflavin may be riboflavin-5′-phosphate (or FMN).

The corneal epithelium is a lipophilic layer of a thickness that can vary from 50 to 100 μM. The corneal epithelium blocks passage of hydrophilic drugs through the corneal epithelium into the cornea, while being relatively passive to lipophilic drugs. Therefore, the hydrophobic molecules, due to their lipophilicity, can permeate the corneal epithelium through the trans-cellular route, in a much more efficient way than hydrophilic molecules. Falvins such as FMN are water-soluble molecules that poorly penetrate through the epithelium and cannot reach the corneal stroma in sufficient amounts to effect treatment. Compositions containing chitosan and a flavin may be capable of penetrating the corneal epithelium without surgical removal of the corneal epithelium, thus allowing delivery of flavins to the cornea without surgical intervention.

The amount of cationic polysaccharides and functionalized cationic polysaccharides in the compositions of embodiments may vary and may be in any therapeutically effective amount. For example, a therapeutically effective amount of flavin in compositions of the invention can be about 0.005% (w/w) to about 10% (w/w), about 0.01% (w/w) to about 9.5% (w/w), about 0.02% (w/w) to about 9.0% (w/w), about 0.03% (w/w) to about 8.5% (w/w), about 0.04% (w/w) to about 8.0% (w/w), about 0.05% (w/w) to about 7.5% (w/w), about 0.06% (w/w) to about 7.0% (w/w), about 0.07% (w/w) to about 6.5% (w/w), about 0.08% (w/w) to about 6.0% (w/w), about 0.1% (w/w) to about 5.5% (w/w), about 0.25% (w/w) to about 5.0% (w/w), about 0.5% (w/w) to about 4.5% (w/w), about 1.0% (w/w) to about 4.0% (w/w), about 1.5% (w/w) to about 3.5% (w/w), about 2.0% (w/w) to about 3.0% (w/w), or any individual concentration or range encompassed by these example ranges.

In some embodiments, the composition may further include one or more quaternary ammonium cations. Quaternary ammonium cations, also known as “quats” or “QACs,” are positively charged polyatomic ions of the structure NR₄⁺, in which R is an alkyl group or an aryl group, and are permanently charged, independent of the pH of their solution. Quaternary ammonium salts or quaternary ammonium compounds are salts of quaternary ammonium cations. The composition of various embodiments can include a quaternary ammonium cation or a quaternary ammonium salt. In some embodiments, each R may, independently be C₂ to C₁₄ alkyl chain, and in particular embodiments at least one R may be a C₅ to C₁₀ aryl or at least one R may include a C₅ to C₁₀ aryl as a substituent on an C₂ to C₁₄ alkyl chain alkyl. Each C₅ to C₁₀ aryl can be substituted with one or more C₂ to C₆ alkyl. For example, in some embodiments, at least one R may be ethylbenzyl. In certain embodiments, the quaternary ammonium salt may be benzalkonium chloride, C₁₀-C₁₄-alkyl(ethylbenzyl)dimethylammonium chloride, and the like or combinations thereof. Combination quaternary ammonium salts can be composed of various ratios of compounds, and the quaternary ammonium of embodiments can include 1^{st} generation, 2^{nd} generation, 3^{rd} generation, 4^{th} generation, 5^{th} generation quaternary ammonium salts or disinfectants. The amount of quaternary ammonium salt in the compositions of embodiments can be from about 0.00005% to about 0.15% (w/w), about 0.001% to about 0.125% (w/w), about 0.001% to about 0.1% (w/w), or any concentration or range of concentrations encompassed by these example concentrations.

The compositions of embodiments can include various additional components known and useful in the ophthalmic arts. For example, the compositions may include an ophthalmic astringent that helps clear mucus from the surface of the eye by precipitating protein. Such ophthalmic astringents include, for example, zinc sulfate and the like. In some embodiments, the compositions may include an ophthalmic demulcent that protects and lubricates mucous membranes such as, for example, cellulose derivatives, carboxymethylcellulose sodium, hydroxyethyl cellulose, hydroxypropyl methylcellulose, methylcellulose, dextran 70, gelatin, liquid polyols, glycerin, polyethylene glycol 300, polyethylene glycol 400, polysorbate 80, polyvinyl alcohol, povidone, and the like and combinations thereof. In some embodiments, the compositions may include ophthalmic emollients, which protects or softens tissues surrounding the eye and to prevent drying and cracking such as, for example, lanolin, anhydrous lanolin, lanolin, oleaginous ingredients, light mineral oil, mineral oil, paraffin, petrolatum, white ointment, white petrolatum, white wax, yellow wax, and the like and combinations thereof. In some embodiments, the compositions may include an ophthalmic hypertonicity agent such as sodium chloride and the like. In some embodiments, the compositions may include an ophthalmic vasoconstrictor such as, ephedrine hydrochloride, naphazoline hydrochloride, phenylephrine hydrochloride, tetrahydrozoline hydrochloride, and the like and combinations thereof. The compositions of various embodiments can include any combination of the above components. For example, in certain embodiments, the compositions may include an astringent and a vasoconstrictor; any 2-3 demulcents; a demulcent and a vasoconstrictor; an astringent, a vasoconstrictor, and a demulcent; and the like.

The compositions of embodiments may further include various solvents and other components that buffer or stabilize the formulation, improve shelf-life, and activate the composition upon delivery. The solvent can be any solvent that does not produce substantial detrimental effect on affected tissue such as the eye or ocular tissue being cared for or irrigated. In certain embodiments, the solvent may be aqueous-based, such as saline, conventional saline solution, or conventional buffered saline solution. The solvent may have a pH in the range of about 5 to about 10, about 5 to about 8, or any individual value or range encompassed by these example ranges. In certain embodiments, the solvent may have ophthalmically acceptable tonicity levels, for example, of at least about 200 mOsmol/kg, or about 200 mOsmol/kg to about 400 mOsmol/kg.

The amount of buffer component employed is preferably sufficient to provide the precursor-containing liquid medium with the desired pH. Under mildly acidic conditions, in particular at a pH of less than about 6 and especially in the pH range of about 3 to about 5, the production of chlorine dioxide is affected from the chlorine dioxide precursors. As such, during chlorine dioxide generation using acid activation, a liquid medium may have a pH of about 6 or less, in particular in the pH range of about 3 to about 5. Any suitable acidic component may be employed as the activator. The primary criteria for such acidic component is that it has the ability to increase the acidity of the liquid medium containing chlorine dioxide precursor sufficiently to effect formation of chlorine dioxide from such chlorine dioxide precursor, and preferably sufficiently to effect formation of antiseptic amounts or disinfecting amounts of chlorine dioxide from the presently useful chlorine dioxide precursors. Such acidic components should also have no substantial detrimental effect on the tissue being cared for. Examples of acidic components include mineral acids, salts of such mineral acids, carboxylic acids, salts of such carboxylic acids and mixtures thereof. The mineral acids include, for example, citric acid, sulfuric acid, hydrogen halides, phosphoric acid and the like. The carboxylic acids include both mono- and poly-, e.g., di-, tri- and the like, carboxylic acids, and preferably include 1 to about 10 carbon atoms per molecule. One or more non-hydrocarbonaceous groups, e.g., hydroxyl groups, halide groups and the like, may be appended to the carboxylic acid. If any acid salt is employed, it is preferred that the salt be an alkali or alkaline earth metal salt, more preferably an alkali metal salt. A particularly useful group of acidic components is selected from alkali metal hydrogen phosphates, citric acid, lactic acid, tartaric acid and mixtures thereof.

Effective amounts of buffer components, tonicity adjusting components, or the combination thereof may be included to provide that such compositions have the desired pH values, tonicities, or the combination thereof. Buffer components, tonicity adjusting components, or the combination thereof useful in other ophthalmic-related compositions may be employed in the presently useful compositions. In addition, one or more other components, such as those known to be useful in ophthalmic-related compositions, may be included in the presently useful compositions in amounts effective to provide such compositions with one or more desired properties. For example, the form of the presently useful compositions may be obtained, maintained, or the combination thereof using one or more of such other components, as fillers, emollients, surfactants, and the like. While the precise regimen is left to the discretion of the clinician, it is recommended that the resulting solution be topically applied by placing one drop in each eye 1 to 24 times daily. For example, the solution may be applied 1, 2, 4, 6, 8, 12, 18 or 24 times a day.

Oxygen-releasing compounds useful as activator components in the present invention include both inorganic and organic peroxy compounds. For example, in some embodiments, the oxygen-releasing compounds such as water soluble inorganic salts such as, for example, the sodium, potassium, calcium, magnesium, lithium and ammonium salts of oxygen-releasing sulfur compounds, such as, for example, the perthiosulfates (S₂O₅.) the persulfates (SO₅⁻²), the peroxysulfates, such as the peroxymonosulfates (HSO₅.) and the peroxydisulfates (S₂O₈⁻²), and combinations thereof, can be used in the combination with chlorine dioxide precursors. In particular embodiments, the oxygen-releasing compound can be potassium peroxymonosulfate (KHSO₅) or a triple salt form of potassium peroxymonosulfate containing potassium peroxymonosulfate (KHSO₅), potassium hydrogen sulfate (KHSO₄) and potassium sulfate (K₂SO₄). This composition is an acidic, water soluble, oxygen releasing powder^{μ} that is odorless, white, granular, stable and free flowing. Other alkali metal, e.g., ^{μ}sodium, and ammonium salts are also useful. Among useful organic peroxy compounds are the aliphatic and aromatic percarboxylic acids. Examples of the aliphatic peracids include peracetic acid, perpropionic acid, up to perlauric acid. The preferred peracids are aromatic such as perbenzoic acid and nuclear substituted perbenzoic acids, especially those having melting points above 50° C. Especially preferred is p-methoxyperbenzoic acid. The amount of oxygen-releasing compound during the chlorine dioxide production may be about 0.01 mole or less to about 1 mole or more per mole of potential chlorine dioxide present as chlorine dioxide precursor in the medium. Particularly useful results are achieved using oxygen-releasing compounds in the range of about 0.01 mole to about 0.1 mole per mole of potential chlorine dioxide present as chlorine dioxide precursor in the medium.

Any suitable activator component may be employed to affect the generation of chlorine dioxide from the presently useful chlorine dioxide precursor components. Examples include acidic materials to increase the acidity of the medium, transition metal components, oxygen-releasing compounds, organic acid anhydrides, chlorine dioxide reducing components and the like. In addition, an electrical current can be passed through a chlorine dioxide precursor-containing liquid medium to effect formation of chlorine dioxide Such compositions may be formulated for topical administration, such as topical administration to the eye. The compositions of embodiments can be formulated as percent by weight solutions in water, saline, or other medical solution.

The compositions of embodiments can further include various additional active agents, such as, anti-inflammatories, steroids, anesthetic, antimicrobial agents, and the like, and combinations thereof. In certain embodiments, the chlorine dioxide or chlorine dioxide precursor containing composition may improve the effectiveness of such active agents. For example, the chlorine dioxide or chlorine dioxide precursor may act as an enhancer for the active agent reducing the necessary concentration to achieve therapeutically effective activity.

In some embodiments, the composition may further include an anti-inflammatory. Numerous anti-inflammatory agents are available in the prior art and any such agents can be used in the compositions of embodiments. For example, suitable anti-inflammatories may include ketotifen fumarate, diclofenac sodium, flurbiprofen sodium, ketorlac tromethamine, suprofen, celecoxib, naproxen, rofecoxib, or a derivative or combination thereof. Ketorolac (also called ketorolac, or ketorolac tromethamine) is a non-steroidal anti-inflammatory drug (NSAID) in the family of propionic acids.

In certain embodiments, the composition may further include a steroid. Numerous steroids are available in the prior art and any such agents can be used in the compositions of embodiments. For example, suitable steroids include, but are not limited to, dexamethasone, dexamethasone alcohol, dexamethasone sodium phosphate, fluorometholone acetate, fluorometholone alcohol, loteprednol etabonate, medrysone, prednisolone, prednisone, prednisolone acetate, prednisolone sodium phosphate, rimexolone, hydrocortisone, hydrocortisone acetate, lodoxamide tromethamine, methylprednisolone, or a derivative or combination thereof.

In further embodiments, the composition may further include an anesthetic. Numerous anesthetics are available in the prior art and any such agents can be used in the compositions of embodiments. For example, suitable anesthetics include proparacaine, lidocaine, tetracaine, and derivatives or combination thereof.

In some embodiments, the composition may further include an antimicrobial agent. Numerous antimicrobial agents are available in the prior art and any such agents can be used in the compositions of embodiments. For example, suitable antimicrobial agents include, but are not limited to, benzalkonium chloride, thimerosal, chlorobutanol, methyl paraben, propylparaben, phenylethyl alcohol, EDTA, sorbic acid, Onamer M (polyquaternium-1), sodium chloride, tyloxapol, sodium sulfate, hydroxyethylcellulose, silver sulfadiazine, colloidal silver, hydrogen peroxide, polyhexamethylene biguanide, myristamidopropyl dimethylamine, and other agents known to those skilled in the art, or a combination thereof. Typically, such preservatives are employed at a level from about 0.001% (w/w) to about 1.0% (w/w).

In some embodiments, the composition may include a vasoconstrictor. Numerous vasoconstrictors are available in the prior art and any such agent can be used in the compositions of embodiments. For example, suitable vasoconstrictors include the following: about 0.01% (w/w) to about 1% (w/w) ephedrine hydrochloride, about 0.001% (w/w) to about 1% (w/w) naphazoline hydrochloride, about 0.001% (w/w) to about 0.5% (w/w) phenylephrine hydrochloride, about 0.001% (w/w) to about 0.5% (w/w) tetrahydrozoline hydrochloride, and the like, or a derivative or combination thereof.

The compositions may be in any suitable form for topical delivery such as a cream, lotion, ointment, gel, liquid, spray and the like, and are preferably administered directly to injured tissue. Such compositions may be in the form of a solution, suspension, or emulsion. In various embodiments, the composition may further include various additives such as, for example, co-solvents, viscosity agents, astringents, demulcents, emollients, hypertonicity agents, preservatives, and combinations thereof.

In particular embodiments, the composition may contain a co-solvent. The solubility of the components of the compositions discussed above may be enhanced by a surfactant or other appropriate co-solvent in the composition. Such co-solvents include, for example, various surfactants, polysorbate 20, 60, and 80, polyoxyethylene/polyoxypropylene surfactants, cyclodextrin, tyloxapol, poloxamer 237 and the like, or combinations thereof. Typically, such co-solvents are employed at a level of from about 0.01% to about 2% by weight.

In some embodiments, the compositions may contain a viscosity agent used to increase viscosity of the composition. Viscosity increased above that of simple aqueous solutions may be desirable to increase absorption of the active compound, to decrease variability in dispensing the composition, to decrease physical separation of components of a suspension or emulsion of the compositions or otherwise improve the composition. Such viscosity agents include, for examples polyvinyl alcohol, polyvinyl pyrrolidone, methyl cellulose, hydroxypropyl methylcellulose, hydroxyethyl cellulose, carboxymethyl cellulose, hydroxypropyl cellulose, and the like, or combinations thereof. Such agents are typically employed at a level of from about 0.01% to about 2% by weight.

In some embodiments, the compositions may include an astringent. Suitable ophthalmic astringents include, for example, zinc sulfate, and the like, or a derivative or combination thereof. Typically, such agents are employed at a level from about 0.005% to about 2% by weight.

In further embodiments, the compositions may further include a demulcent. Suitable demulcents include, for example, about 0.05% (w/w) to about 5% (w/w) carboxymethylcellulose sodium, about 0.05% (w/w) to about 5% (w/w) hydroxyethyl cellulose, about 0.05% (w/w) to about 5% (w/w) hydroxypropyl methylcellulose, about 0.05% (w/w) to about 5% (w/w) methylcellulose, about 0.005% (w/w) to about 1.0% (w/w) Dextran 70, about 0.005% (w/w) to about 1.0% (w/w) Gelatin, about 0.005% (w/w) to about 5.0% (w/w) Glycerin, about 0.005% (w/w) to about 5.0% (w/w) polyethylene glycol, about 0.005% (w/w) to about 1.0% (w/w) polysorbate, about 0.005% (w/w) to about 10% (w/w) polyvinyl alcohol, about 0.005% (w/w) to about 5.0% (w/w) povidone, or a derivative or combinations thereof.

In another embodiment, the composition may further include emollients. Suitable emollients include, for example, about 0.1% (w/w) to about 20% (w/w) anhydrous lanolin, about 0.1% (w/w) to about 20% (w/w) lanolin, about 0.005% (w/w) to about 50% (w/w) mineral oil, about 0.005% (w/w) to about 10% (w/w) paraffin, about 0.005% (w/w) to about 100% (w/w) petrolatum, about 0.005% (w/w) to about 100% (w/w) white ointment, about 0.005% (w/w) to about 100% (w/w) white petrolatum, about 0.005% (w/w) to about 10% (w/w) white wax, about 0.005% (w/w) to about 10% (w/w) yellow wax, or derivatives or combinations thereof.

In further embodiments, the composition may include a hypertonicity agent. Suitable hypertonicity agents include the following: about 0.1% (w/w) to about 10% (w/w) sodium chloride, or derivatives or combinations thereof.

In some embodiments, the composition may include a preservative. Suitable preservatives include, for example, benzalkonium chloride, polyquad, sodium perborate, stabilized oxychloro complex, ocupure, polyhexamethylene biguanide, chlorobutanol, edetate disodium, polyaminopropyl biguanide, polyquaternium, or derivatives or combinations thereof.

In some embodiments, the composition may further include sorbitol, hyaluronic acid, sodium hyaluronate, carnitine, erythritol, hydroxypropyl guar, polyacrylic acid, tyloxapol, tromethamine, or derivatives or combinations thereof. In some embodiments, the composition may further include hyaluronan, sulfobetaine, poloxamine, boric acid, sodium borate, edetate disodium, sodium chloride, hydroxyalkylphosphonate, propylene glycol, or derivatives or combinations thereof.

In some embodiments, the composition may further include hydrogen peroxide, lactic acid, halogen salts, salts of heavy metals, chlorhexidine, dodecyl-methyl-polyoxy-ethyl-ammonium propionate, chlorides of ammonium, ammonium propylamide, lauryl sulphate, dodecyl sulphate, alkyl succinic salts, ethyl alcohol, isopropyl alcohol, chlorhexidine, non-chlorinated quaternary ammonium salts, chlorinated quaternary ammonium salts, dodecyl-methyl-polyoxy-ethyl-ammonium propionate, iodine, benzyl dimethyl ammonium chloride, sodium tetraborate decahydrate, terric GN9, dipropylene glycol methyl ether, alkali metals, alkaline earth metals, hypochlorite, sodium hydroxide, or derivatives or combinations thereof.

The compositions embodied by the description above can be formulated as a solution for ophthalmic delivery and the methods may include administering a drop of the solution to an eye, using any of the many types of eye drop dispensers on the market. Administering can include periodic or repeated applications of the chlorine dioxide containing composition to the eye. Such periodic applications to eyes, which are susceptible to, but do not have, microbial infections, may ameliorate or prevent microbial infections in the eyes. In certain embodiments, the dosage for one eye may be about one drop of solution, which can include about 10 μl (microliters) to about 200 μl, about 20 μl to about 120 μl, or about 50 μl to about 80 μl of solution, or any values in between. For example, dispensers such as pipettors can dispense fluid drops from at least 1 μl to 300 μl, and any value in between.

Additional embodiments are directed to methods for using the compositions described above including chitosan and a cationic polysaccharide or functionalized cationic polysaccharide.

Such methods include contacting an eye of a subject with a composition of embodiments after corneal transplantation. A corneal transplantation procedure (keratoplasty) involves replacing the diseased or damaged tissue of a patient's cornea with a graft of healthy tissue that is taken from a donor cornea. Corneal transplantation procedures have been generally performed using either a knife or some form of laser procedure to prepare the patient's cornea and create a donor graft. In such embodiments, the compositions of embodiments may be administered to the cornea of the subject during corneal transplantation, and in other embodiments, the compositions of embodiments may be administered to the cornea of the subject after corneal transplantation, when the underlying cornea is covered by the corneal epithelium cannot be contacted directly by the composition. In such embodiments, the chitosan may allow for penetration of the cationic polysaccharide or functionalized cationic polysaccharide into the cornea where it can cause crosslinking of the cornea and the corneal epithelium without surgically exposing the cornea. Thereby, providing treatment, while reducing exposure of the cornea to the environment.

Other embodiments include methods for treating keratoconus or other ectasic corneal disorders, such as, post lasik ectasia, keratectasia, progressive myopia, and glaucoma with the compositions described above. Keratoconus is a degenerative disease of the eye in which structural changes within the cornea cause it to thin and change to a more conical shape than its normal gradual curve. Such methods may generally include contacting an eye of a subject with a composition of embodiments. In such embodiments, chitosan may enhance the permeability of the corneal epithelium, allowing flavin to penetrate the corneal epithelial layer without having removed the epithelium during the crosslinking procedure. The removal of the epithelium during crosslinking is referred to as the “epi-off technique.” When the epithelium is not removed the technique is referred to “epi-on.”

1. A composition comprising an effective amount of chitosan, an effective amount of a flavin, and a pharmaceutically acceptable excipient, carrier, or diluent.

2. The composition of claim 1, wherein chitosan comprises acetylated or deacetylated chitosan, acetylated or deacetylated chitosan salts, acetylated or deacetylated chitin, acetylated or deacetylated chitin sats, acetylated or deacetylated chitooligosaccharides, acetylated or deacetylated chitooligosaccharides salts, acetylated or deacetylated functionalized chitosan derivatives, acetylated or deacetylated functionalized chitosan derivative salts, N,N,N-trimethyl chitosan (TMC), N,N,N-trimethyl-O-(2-hydroxy-3-trimethylammonium propyl)chitosan, N,N,N-trimethyl-O-carboxymethyl chitosan (TMCMC), O-carboxymethyl chitosan (CMC), sodium-form O-carboxymethyl chitosan, hydorgen-form O-carboxymethyl chitosan, N-(2-hydroxy-3-trimethylammonium)propyl chitosan (HTCC), quaternary N-(benzyl)chitosan, and combinations thereof.

3. The composition of claim 1, wherein an effective amount of chitosan comprises about 10% (w/w) to about 75% (w/w) based on the total weight of the composition.

4. The composition of claim 1, wherein the chitosan is a thiolated polymer, thiomer, or preactivated thiomer selected from the group consisting of chitosan-cysteine, chitosan-thiogylcolic acid, chitosan-thiobutylamidine, deacetylated gellan gum-cysteine, poly(methacrylic acid)-cysteine, alginate-cysteine, carboxymethycellulose-cysteine, poly(acrylic acid)-cysteine, poly(acrylic acid)-cysteamine, poly(acrylic acid)-homocysteine, and thiolated polycarbophil, and combinations thereof

5. The composition of claim 1, wherein the flavin is selected from the group consisting of riboflavin, riboflavin-5-phosphate, flavin mononucleotide, flavin adenine dinucleotide, flavin guanine nucleotide, flavin cytosine nucleotide, flavin thymine nucleotide, riboflavin free base, phosphorylated riboflavin, riboflavin-5′-phosphate, or salts or derivatives thereof, and combinations thereof.

6. The composition of claim 1, wherein an effective amount of flavin comprises about 0.005% (w/w) to about 10% (w/w) based on the total weight of the composition.

7. The composition of claim 1, further comprising an effective amount of one or more quaternary ammonium cations.

8. The composition of claim 7, wherein the one or more quaternary ammonium cations are selected from the group consisting of benzalkonium chloride, C₁₀-C₁₄-alkyl(ethylbenzyl)dimethylammonium chloride, andor salts or derivatives thereof, and combinations thereof.

9. The composition of claim 7, wherein the effective amount of one or more quaternary ammonium cations comprises 0.00005% (w/w) to about 0.15% (w/w) based on the total weight of the composition.

10. The composition of claim 1, further comprising one or more solvents, co-solvents, buffers, stabilizers, ophthalmic astringents, ophthalmic demulcents, ophthalmic emollients, ophthalmic vasoconstrictors, oxygen-releasing components, activators, hypertonicity agents, and combinations thereof.

11. The composition of claim 1, further comprising one or more anti-inflammatory agents, steroids, anesthetics, antimicrobial agents, chlorine dioxide, and combinations thereof.

12. A method for treating corneal disorders comprising administering an effective amount of a composition comprising chitosan, flavin, and a pharmaceutically acceptable excipient, carrier, or diluent to an eye of a patient in need of treatment.

13. The method of claim 12, wherein administering comprises periodic or repeated application of the composition to the eye of the patient in need of treatment.

14. The method of claim 12, wherein an effective amount of the composition comprises about 10 μl (microliters) to about 200 μl.

15. The method of claim 12, wherein the patient in need of treatment has undergone a corneal transplant, has keratoconus, ecstatic corneal disorder, post lasik ectasia, keratectasia, progressive myopia, glaucoma, and combinations thereof.

16. The method of claim 12, wherein chitosan comprises acetylated or deacetylated chitosan, acetylated or deacetylated chitosan salts, acetylated or deacetylated chitin, acetylated or deacetylated chitin sats, acetylated or deacetylated chitooligosaccharides, acetylated or deacetylated chitooligosaccharides salts, acetylated or deacetylated functionalized chitosan derivatives, acetylated or deacetylated functionalized chitosan derivative salts, N,N,N-trimethyl chitosan (TMC), N,N,N-trimethyl-O-(2-hydroxy-3-trimethylammonium propyl)chitosan, N,N,N-trimethyl-O-carboxymethyl chitosan (TMCMC), O-carboxymethyl chitosan (CMC), sodium-form O-carboxymethyl chitosan, hydorgen-form O-carboxymethyl chitosan, N-(2-hydroxy-3-trimethylammonium)propyl chitosan (HTCC), quaternary N-(benzyl)chitosan, and combinations thereof.

17. The method of claim 12, wherein the chitosan has a concentration of about 10% (w/w) to about 75% (w/w) based on the total weight of the composition.

18. The method of claim 12, wherein the flavin is selected from the group consisting of riboflavin, riboflavin-5-phosphate, flavin mononucleotide, flavin adenine dinucleotide, flavin guanine nucleotide, flavin cytosine nucleotide, flavin thymine nucleotide, riboflavin free base, phosphorylated riboflavin, riboflavin-5′-phosphate, or salts or derivatives thereof, and combinations thereof.

19. The method of claim 12, wherein the flavin has a concentration of about 0.005% (w/w) to about 10% (w/w) based on the total weight of the composition.

20. The method of claim 12, wherein the composition further comprises an effective amount of one or more quaternary ammonium cations.

21. The method of claim 12, wherein the composition further comprises one or more solvents, co-solvents, buffers, stabilizers, ophthalmic astringents, ophthalmic demulcents, ophthalmic emollients, ophthalmic vasoconstrictors, oxygen-releasing components, activators, hypertonicity agents, and combinations thereof.

22. The method of claim 12, wherein the composition further comprises one or more anti-inflammatory agents, steroids, anesthetics, antimicrobial agents, chlorine dioxide, and combinations thereof.
